(19) United States
(12) Patent Application Publication
(10) Pub. No.: US 2011/0027818 A1
(43) Pub. Date: Feb. 3, 2011

(54) APPARATUS AND METHOD FOR DETECTING ZINC IONS

(75) Inventor: Christopher J. Frederickson, Galveston, TX (US)

Correspondence Address: Don D. Cha, 225 Union Blvd, Suite 150, Lakewood, CO, 80228, US

(73) Assignee: ANDRO DIAGNOSTICS, Galveston, TX (US)

(21) Appl. No.: 12/901,186

(22) Filed: Oct. 8, 2010

Related U.S. Application Data
(63) Continuation-in-part of application No. 11/221,167, filed on Sep. 7, 2005, now abandoned.
Application No. 11/221,167 is a continuation-in-part of application No. 10/829,732, filed on Apr. 22, 2004.

(51) Int. Cl. C12Q 1/02

(52) U.S. Cl. 435/29

(57) ABSTRACT

The present invention provides apparatuses and methods for determining zinc ion concentration in a fluid sample. Some apparatuses of the invention include (i) a light source designed to emit an excitatory light at a first known wavelength that activates a fluorescent emission having a second and different wavelength by at least one selected fluorescent reporter probe that generates a different fluorescent response when bound to zinc ion; (ii) at least one sample-holding component adapted to hold a liquid sample in a pathway of the excitatory light; (iii) a photodetector for measuring fluorescent emission intensity generated by fluorescent reporter probe bound to zinc ion within the liquid sample; and (iv) electronic means for creating a data signal that correlates with fluorescent emission intensity generated by fluorescent reporter probe bound to zinc ion in the liquid sample to the free zinc ion concentration. Such apparatuses and methods can be used to determine the presence of or the risk of having prostate cancer in a subject.

### CROSS-REFERENCE TO RELATED APPLICATIONS

This application is a continuation-in-part of U.S. patent application Ser. No. 11/221,167, filed Sep. 7, 2005 and U.S. patent application Ser. No. 10/829,732, filed Apr. 22, 2004, which are incorporated herein by reference in their entirety.

### FIELD OF THE INVENTION

This invention relates to devices for detecting zinc ions and methods for using the same, for example, to determine the presence of prostate cancer or the risk for prostate cancer in an individual.

### BACKGROUND OF THE INVENTION

In various fields of biochemistry and medicine, there is a growing recognition that concentrations of zinc in biological liquids are highly important. Of particular importance are concentrations “free zinc.” As used herein, the term “free zinc” refers generally to zinc ions that are not bound to proteins, peptides, or amino acids. The term “free zinc” also excludes zinc ions that are inside cells. More accurately, the term “free zinc” refers to zinc having a dissociation constant (K_{D}) of about 10⁻⁴ or higher, typically, 10⁻² or higher, and often 1 or higher.

Zinc is an abundant transition metal in the human body. In a normal male reproductive system semen has about 3 mM of zinc, which is approximately 1000-fold more than those found in saliva, tears, vaginal secretions, urine or blood. Indeed, ejaculate contains so much zinc that a zinc-sensitive dye has been proposed for use by police to find semen at crime scenes. Why zinc is present in semen has not been established clearly. Regardless of the function of zinc in semen, the source of zinc appears to be in a small part from the testes, which concentrates zinc in and on the spermatozoa, but primarily from the secretory cells lining the ducts of the lateral lobes of the prostate gland.

The present inventor has discovered that semen zinc level is a sensitive and selective prostate cancer indicator. Unfortunately, measuring zinc in complex biological matrices such as semen and determining the sizes of the different “pools” of zinc and the changes, if any, in these multiple zinc pools is a daunting bioanalytic problem.

Therefore, there is a need for a device for easily measuring the distribution, speciation and concentrations of zinc in prostate tissue and seminal fluid.

### SUMMARY OF THE INVENTION

Some aspects of the invention provide a device or an apparatus that allows simple, rapid, and convenient method of measuring free zinc concentrations in fluid samples. Such devices and apparatuses provide simple methods for determining the presence or the risk of prostate cancer in a subject when the fluid sample comprises semen, expressed prostatic fluid, VB3 urine, or a mixture thereof.

In one particular embodiment, the apparatus of the invention comprises:
a light source designed to emit an excitatory light at a first known wavelength that activates a fluorescent emission having a second and different wavelength by at least one selected fluorescent reporter probe that generates a different fluorescent response when bound to zinc ion;
at least one sample-holding component adapted to hold a liquid sample in a pathway of the excitatory light;
a photodetector for measuring fluorescent emission intensity generated by fluorescent reporter probe bound to zinc ion within the liquid sample; and
electronic means for creating a data signal that correlates with fluorescent emission intensity generated by fluorescent reporter probe bound to zinc ion in the liquid sample to the free zinc ion concentration.

Apparatuses of the invention can be used to determine the presence of prostate cancer or the risk for prostate cancer in an individual. Such a determination can be made by a method comprising:
determining an amount of free zinc in a fluid sample of an individual using a zinc ion detection apparatus disclosed herein, where the fluid sample comprises semen, expressed prostatic fluid, VB3 urine, or a mixture thereof; and
determining the presence of prostate cancer or the risk for prostate cancer in the individual using the amount of free zinc determined from the fluid sample of the individual.
   The zinc ion detection apparatus determines the zinc ion concentration in the fluid sample excluding zinc ions that are bound to proteins, peptides, amino acids and the zinc ions in spermatozoa or endothelial cells that maybe present in the fluid sample.

### BRIEF DESCRIPTION OF THE DRAWINGS

FIG. 1 is a schematic illustration of the arrangement of the main components of a pZn metering device as disclosed herein, showing an enclosure box that holds a light source, a first electromagnetic wave filter, a square cuvette for holding a fluid sample, a second electromagnetic wave filter, an electronic photodetector for measuring fluorescent light intensity, an optional digital display panel, an internal microprocessor and memory chip, and a computer interface.

FIG. 2 is a schematic illustration of one embodiment of a system of this invention comprising a metering device in combination with supply of cuvettes, a supply of at least one fluorophore reagent, a set of sealed calibration cuvettes containing known zinc concentrations, and a supply of an ion-binding reagent that chelates other divalent metals, such as copper, nickel, cadmium, etc.

FIG. 3 is a graph of a calibration curve that was plotted for the ZP1 fluorophore reagent, showing pZn values on the horizontal axis, and fluorescence intensity values (in arbitrary units, derived from the number of “counts” measured by a charge-coupled photodetector inside a zinc meter) on the vertical axis.

### DETAILED DESCRIPTION OF THE INVENTION

Some aspects of the invention provide apparatuses that allow simple and convenient measurement of free zinc concentrations. Such apparatuses provide accurate and reliable measurement with minimum interference and inaccuracies caused by potentially competing elements, including potentially divalent metals such as copper, iron, nickel, cadmium, mercury, etc. Some apparatuses of the invention use a metering device that uses fluorescent emissions that are altered when a fluorescent reporter probe binds to free zinc. Such apparatuses can be coupled to a computer that can help an operator perform various adjustments and calculations. For example, measured fluorescence can be used to correlate to a free zinc concentration that can be used to determine certain traits and relevance.

In some embodiments, apparatuses of the invention are a self-contained machine or device that include receive and hold a sample holder such as cuvettes or other suitable vessels. In some instances apparatuses of the invention can also include a calibration sample holder that can hold a vessel containing a known concentration of zinc. Such a calibration sample can be used to calibrate and adjust the measuring apparatus to ensure accuracy and reliability.

Still in other embodiments, apparatuses of the invention can be connected to or include a computer or a microprocessor and a display system.

Apparatuses of the invention can rapidly measure concentrations of free zinc in samples of liquid (such as blood, cerebrospinal fluid, cell culture media, seminal fluid, etc.). In some embodiments, apparatuses of the invention use a fluorescent reporter probe comprising a fluorophore that fluoresces at a different wavelength when bound to zinc ion.

A fluorophore typically absorbs light at a certain wavelength and reemits light at a different wavelength. The emitted (i.e., fluorescent) light is generally of a longer wavelength than the absorbed light. For example, many fluorophores absorb ultraviolet (UV), near-UV, or blue light and emit a green, yellow, or other longer wavelength light. In some embodiments, instead of fluorescing at wavelengths that were not previously present before binding to zinc ion, some fluorescent reporter probes emit light at increased intensity in a fluorescent color that was being emitted by that same fluorophore before binding to zinc ion. As a result, conventional types of metering device as known in the art cannot simply correlate a color intensity, with a zinc ion concentration. In these instances, additional steps must be taken to correlate the color intensity to the zinc ion concentration.

There are a number of fluorophores that result in measurable alterations when bound to zinc ion. As stated herein, a fluorophore reporter probe comprises a fluorophore moiety, which is the moiety responsible for fluorescence. Often the terms “fluorophore reporter probe” and “fluorophore” are used interchangeably herein. However, it should be appreciated that unless the context requires otherwise, the term “fluorophore” refers to “fluorophore reporter probe.” Exemplary fluorophore reporter probes that are useful include, but are not limited to, zinpyr compounds, which contain a ring structure derived from pyridine. Several compounds in the zinpyr class are known by common designations such as ZP1, ZP4, and ZP8, since their chemical names are long and complex (for example, the full name of the ZP4 compound is 9-(o-carboxy-phenyl)-2-chloro-5-[2-(bis(2-pyridylmethyl)-aminomethyl)-N-m-ethylaniline]-6-hydroxy-3-xanthanone)). These fluorophores well known to one skilled in the art. These fluorescent reporter probes display an increased level of quantum efficiency (which is also referred to by terms such as photon efficiency, photon emission efficiency, emission efficiency, fluorescence efficiency or intensity, etc.) when bound to zinc ion. For example, a zinpyr fluorophore preparation might display a quantum efficiency of about 0.3 in the absence of any free zinc. This means that if that particular reagent is activated by a certain quantity or intensity of activation energy at a suitable wavelength (such as 480 nm), it will emit roughly 30% of that same number of photons, but at a different wavelength, such as 530 nm). By contrast, if the same fluorophore binds to free zinc, its quantum efficiency will increase. Quantum efficiencies of some fluorophores have been shown to increase to a level of about 0.5, while quantum efficiencies of other fluorophores can increase to levels as high as about 0.9. Other useful fluorophore receptor probes include a class of fluorophores known as ZnAF's (e.g., ZnAF-1, ZnAF-2, ZnAF-1F, ZnAF-2F, etc.). See, for example, Wang et al., J. Mater. Chem., 2005, 15, 2836-2839 and references cited therein, which are incorporated by reference in their entirety. These and many fluorophores are photoinduced electron transfer (PET) sensors. In embodiments, fluorophores are PET sensors. Within these embodiments, in some instances fluorophores are ZnAF, and often fluorophores are ZnAF-2.

As can be expected, various fluorophores within the zinpyr class have different properties. For example, the ZP1 compound is relatively hydrophobic and lipophilic; this limits its solubility in aqueous solutions, but it has a relatively high ability to permeate into and through cell and organelle membranes. In contrast, the ZP4 compound is hydrophilic; it is readily soluble in aqueous solutions, but it has lower levels of permeation through lipid membranes.

Other known zinc-selective fluorophores include “zinspy” compounds, which are a sulfur-containing compounds (usually in the form of a thioether-pyridyl derivative). Some members of the zinspy class of fluorophores have improved selectivity for zinc, compared to members of the zinpyr class of compounds; however, zinspy compounds tend to be more water-soluble than various zinpyr compounds, and the reduced lipophilic levels of zinspy compounds reduces their ability to permeate through cellular membranes. Additional classes of fluorophores are known to one skilled in the art including fluorophores that generate fluorescent emissions with increased intensity and efficiency.

In some embodiments of the invention, the quantum efficiency of fluorophores increases when bound to zinc. If desired, alternate fluorophores can be developed in which binding to zinc will reduce, rather than increase, quantum efficiency.

Each fluorophore have its own unique properties, for example, different quantum efficiency, water solubility, ability to permeate into cell membranes, and selectivity for zinc compared to copper, iron, or other metals, etc. These properties can vary substantially among different fluorophores. Therefore, steps must be taken to provide methods and means for ensuring that measurements made with any of various different fluorophores will nevertheless be reliable and accurate. This is accomplished in part by creating a specific response curve for each specific type of fluorophores that is used.

Regardless of which particular fluorophore is used, typically an excess amount of fluorophore is used relative to the amount of free zinc in the sample. This ensures that the number and intensity of fluorophore-zinc complexes that is produced in the sample will depend on the quantity of free zinc in the liquid rather than on the quantity of the fluorophore. If the measured intensity of light emitted at the fluorescing wavelength approaches a saturation value, or if there is any other reason to suspect that the free zinc in a certain sample has saturated or nearly saturated the binding capacity of the fluorophore, it is a simple matter for a user to dilute the liquid that is being tested. This type of dilution is well known to researchers, and it most commonly uses one or a series of 10× dilutions (1 volume of test liquid is mixed with 9 equal volumes of diluent, such as distilled deionized water). If measurements are being made at extremely low levels (such as picomolar or femtomolar concentrations), a supply of diluent liquid that is reliably known to contain no detectable level of free zinc should be used.

While any fluorophore receptor probe can be used, in some embodiments fluorophores that are suited for use have both: (1) a “baseline” (or starting, unaltered, unbound, etc.) value for its quantum efficiency (also called fluorescence efficiency, emission efficiency, etc.) when no zinc ion is present; and (2) a substantially higher quantum efficiency, when the fluorophore become bound to zinc ions in a liquid. In these embodiments, if an excess of fluorophore (relative to zinc ion) is mixed with a liquid sample, the extent of the increase in fluorescent emissions by the zinc-fluorophore complex mixture will be limited and controlled by the amount of zinc ion that was available to react with the excess fluorophore. This can provide a reliable indicator of how much zinc ion was present in the sample to react with the excess fluorophore in the mixture.

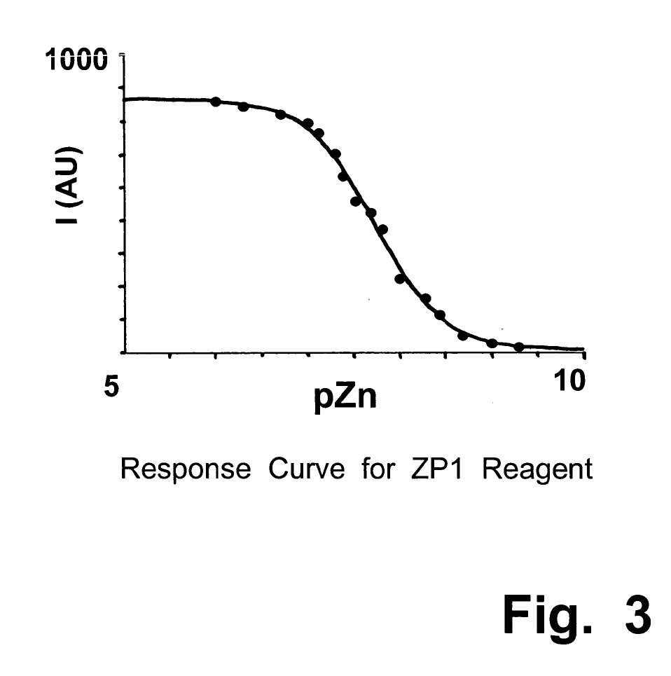

It should be appreciated that different fluorophore reagents have different baseline (unbound) quantum efficiencies and different increase in quantum efficiencies when bound to zinc ion. Therefore, different fluorophore reagents typically generate different response curves. An example of a response curve for the ZP1 fluorophore is provided in FIG. 3. Fluorescence intensities are plotted on the Y axis. These numbers indicate the number of “counts” measured by a photodetector of the apparatus of the invention. Zinc ion concentrations are plotted on the X-axis. The pZn numbering system is directly comparable to the pH system; i.e., it is a negative logarithmic number, base 10. As examples, a pZn value of 3 indicates zinc ion concentration of 10⁻³ and a pZn value of 6 indicates zinc ion concentration of 10⁻⁶, which is a thousand times lower. Since base 10 logarithmic values are used zinc ion concentrations over a very wide range can be conveniently displayed using short and convenient numbers in a manner similar to pH values, e.g., ranging from millimolar (10⁻³), femtomolar (i.e., 10⁻⁵), micromolar (10⁻⁶), nanomolar (10⁻⁹), to picomolar (10⁻¹²). Since higher concentrations of zinc ion will generate greater fluorescent emissions, and since higher concentrations of zinc ion are closer to the vertical axis (i.e., higher concentrations correspond to lower pZn values), a response curve will decrease from left to right similar to that shown in FIG. 3. Response curves for various different fluorophores are well known or can be readily determined by one skilled in the art. If desired, response curves can also be produced by calculations that take baseline versus zinc-bound “quantum efficiency” levels into account.

Once a response curve is known for a particular fluorophore of interest, the apparatus of the invention can be configured or adapted to accommodate and convert any measurements that are made using that particular fluorophore.

If desired, any number of different response curves can be programmed into or stored within the apparatus of the invention. In some embodiments, such apparatuses can automatically calculate and display both: (i) the fluorescence intensity that was measured by the photodetector, and (ii) the zinc ion concentration response curve for that particular fluorophore. If desired, such an apparatus of the invention can prominently display the name of the fluorophore that was used, and it can ask the operator to confirm that selection, to ensure that the proper response curve was selected, for converting a fluorescence intensity measurement into a calculated zinc ion concentration. If desired, the supporting program or software can also be programmed to record the date and time of each measurement, the initials of the researcher who ran the test, and/or the serial or other identifying number of the apparatus. Although that information likely will never be needed, it may become useful, if questions arise at a later date concerning apparent inconsistencies or unexpected observations, especially in research projects involving multiple researchers and technicians.

Under some unusual conditions, the performance of some fluorophores may be affected, to an extent, by the type of liquid being analyzed. Although a specific fluorophore will have generally consistent responses in most types of carrier liquids, differences can arise in how a particular liquid handles activating light and fluorescent emissions, when liquids as different as cerebrospinal fluid versus wastewater effluent are being analyzed. This can become especially important when a liquid contains cells, suspended particles, or other components that may absorb or scatter light at activation or fluorescent wavelengths, or that otherwise create turbidity, opacity, cloudiness, or other factors that influence the behavior or fate of photons passing through the liquid.

Accordingly, response curves using a specific fluorophore in different types of liquid can be created, if a need arises or if desired. For liquids that are of widespread interest and that are relatively constant and predictable (such as human blood serum or plasma, cerebrospinal fluid, urine, semen, saliva, sweat, etc.), response curves can be published, posted on a website, or otherwise made available by one skilled in the art, if the response curve in any particular type of liquid varies significantly from similar curves in other liquids. For other liquids (such as wastewater effluents), response curves can be readily generated by employees, or by contractors or consultants experienced in this type of work, simply by using an apparatus of the invention (e.g., a “pZn meter”) to test samples of the specific liquid being analyzed, after controlled concentrations of zinc chloride or another suitable zinc salt has been mixed with those liquids.

If desired, components can be provided to enable partial or even complete stand-alone operation, by an apparatus of the invention that is not connected to a computer. For example, response curves for various fluorophores can be programmed into a microprocessor that has been selected to provide sufficient memory capacity inside the apparatus. This type of programmable information should be capable of revision and replacement by an operator who has proper authority to update the software that is loaded into the apparatus. This type of approach can allow, for example, raw data indicating the number of counts that are being detected by a photodetector, and the corresponding zinc concentration in the sample that is being measured (based on a known type of fluorophore) to be displayed on a display unit that can be provided on the apparatus if desired. The display unit, in conjunction with a knob, button, or other control device on the surface of the apparatus, also can allow an operator to scroll through an assortment of different response curves stored in the apparatus to select a response curve that corresponds to a particular fluorophore.

In other embodiments, apparatuses of the invention are able to transfer data to a computer, such as by means of a “universal serial bus” (USB) port. USB interfaces (which include inexpensive hubs, routers, and cables) have been designed to provide standardized means for allowing a computer to interact with dozens of peripheral devices. A USB cable also can function as the power cord for the apparatus, since USB cables can provide DC current to drive peripheral devices that do not require substantial power.

Alternately, if desired, a “Firewire” interface, a serial, parallel, or Ethernet cable, a wireless system that uses infrared, radiofrequency, or other emissions, or any other comparable system that is already known or hereafter developed, can be used to transfer data from the apparatus of the invention to a computer, microprocessor, or other devices.

The components that create and provide the apparatus of the invention as described herein can also be combined with other compatible systems. For example, means can be provided for using a single apparatus to carry out measurements of two, three, or more parameters that may be of interest in various biological fluids (such as pH and/or pO2 levels, concentrations of various other elements such as iron, copper, sodium, calcium, etc.). Any such measurements can be carried out by using already known or hereafter discovered means and methods, which can be provided by a larger and more complex system that can include and incorporate a zinc-measuring apparatus as described herein.

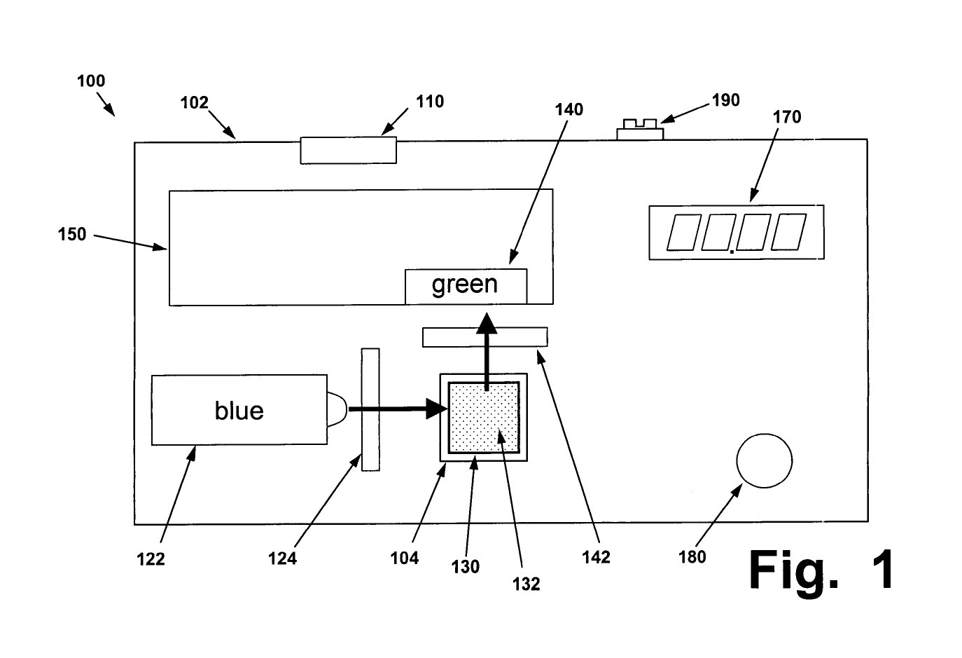

One embodiment of apparatuses of the invention is schematically illustrated in FIG. 1. It should be appreciated that accompanying drawings are mere illustrations of some of the apparatuses of the invention. However, it should be appreciated that the scope of the invention is not limited to these particular embodiments. These drawings are merely illustrative and do not limit the scope of the present invention. Referring to FIG. 1, some aspects of the invention provide an apparatus 100 for measuring concentrations of zinc ion in liquids. Apparatus 100 is also referred to herein as a “meter” or a “pZn meter”, wherein the prefix “p” is used in the same way as the “p” prefix in other chemical measurements, such as pH (for acidity), pO2 (for dissolved oxygen concentrations in liquids), etc.

Meter 100 has at least one support and enclosure component 102, which will hold, enclose, and protect various internal components, including several optical components that are spaced adjacent to each other in controlled locations, as discussed below. Support and enclosure component 102 (which also can be referred to as a box, shell, frame, or similar terms) can be made of one or more pieces of shaped metal, molded plastic, etc. For example, in a typical mode of construction, an internal frame to which the optical and electronic components can be screwed or bolted (allowing convenient removal and replacement of specific parts) is made from strips of aluminum, thin-gauge steel, molded plastic, or other suitable material. The outer box or enclosure generally has a top shell component and a bottom shell component, both of which can be screwed to the frame to allow either or both of them to be removed, and to provide access to the interior components for purposes such as cleaning, repair, replacement, calibration, etc. The meter typically is also provided with rubberized feet or pads on the bottom to help reduce vibrations or other unwanted motion, and to help protect the unit from spills and chemicals.

Typically, meter 100 includes a sample-holding component 104 that can allow sample holders (such as cuvettes) to be easily inserted and removed. Unless the context requires otherwise, the term “cuvette” is used broadly herein to include any type of sample holder e.g., cup-shaped, tube-shaped, or similar device, designed to hold a small quantity of a liquid 132 (which can also be called an analyte, sample, specimen, or similar terms). The walls of a sample holder are typically made of a material that is transparent to both the excitatory light and the emitted fluorescent light. Cuvettes, which are often used as sample holders in a various analytical instruments, typically hold about 1 to about 5 milliliters of liquid, and may be etched with a horizontal line to indicate a proper filling depth; however, measurements of very small quantities of liquids can be important, and specialized types of cuvettes or other sample-holders are known that will allow measurements of only a few microliters. Cuvettes usually are typically made of a clear plastic (such as polycarbonate, which is relatively resistant to heat, acids, etc.), quartz, or glass, and they usually are sold and shipped in boxes of 50, 100, or more.

Cuvettes conventionally have square cross-sectional shapes when seen from above (in a “plan view”). Square shapes can help ensure more consistent readings than round tubes, by causing an excitatory light to pass in a relatively uniform manner through a liquid sample having a consistent thickness, rather than passing through a sample holder having a relatively different thickness in the middle compared to the sides (as would occur if circular tubes were used). If desired, a lens can be provided between light source 122 and cuvette 130, to ensure that the excitatory light passes through the cuvette in a direction that is essentially linear and parallel, with little or no “spread”. If a conventional cuvette-holding component is provided in meter 100, other types of liquid sample holders (such as hollow needles, parallel slides, etc.) can be adapted to fit into the cuvette holder.

Sample holder 104 and fluorescing light detector 140 are typically surrounded by opaque materials to prevent ambient light from reaching those two components in ways that could lead to false readings; therefore, as a matter of convenient design, support and enclosure component 102 should enclose the entire apparatus. This also helps to protect the electronic and other internal components against spills, dust, breakage, etc. The entire closure shell can be made of opaque material, such as extruded aluminum or sheet metal, or opaque molded plastic.

Sample holder 104 is typically positioned beneath a lid, door, sliding panel, or similar access means 106 that can be opened and closed. This access means allows convenient insertion and removal of samples or other devices or components while also keeping ambient light out of the apparatus while the fluorescence of a sample is being measured. The movable access means 106 can use a hinged, sliding, or axle-mounted plate, or any other suitable mechanism. For example, FIG. 1 shows a cover plate 106 that slides on rails 108. The periphery of the lid or door should be provided with a gasket or seal made of a dark resilient material, such as a foam rubber, piled fabric, flexible cylinders of fabric or rubber, etc.

Electrical power is supplied to the apparatus to power light emitter and photodetector. This can be done by a conventional power cord; such conventional power cords can carry either (i) 110 volt AC power (or comparable AC voltage at other voltage levels that may be used in other countries), or (ii) lower voltage AC or DC power, supplied by a transformer that can be plugged in to a wall socket. Alternately, since the device will consume only a relatively small amount of power, low voltage direct current can be supplied by batteries, a USB cable, or similar means (a USB connector or port 110 is shown in FIG. 1). A power cord, USB port, or other electrical supply component can be mounted at any convenient location, such as on either side or the back of the box.

If a USB cable or similar system is used to provide low-voltage power, a capacitor and/or rechargeable battery can be provided as part of the power-handling system inside the apparatus. The capacitor or battery can be charged up between readings, and can provide most of the power that will run the light source and photodetector during a measurement, so the apparatus will not impose abrupt drains on the power supply. However, it should also be noted that USB hubs and routers having multiple ports are available with power supplied by means of a cord plugged directly into a standard outlet. The use of such devices, which eliminate the need for a computer to provide powering voltages to peripherals, can avoid any risk that a sudden power drain by a metering device or other peripheral might cause a computer malfunction.

Meter 100 contains an optical system, which includes at least one light source 122 (such as a bulb with a heated filament, a light-emitting diode, etc.) and at least one photodetector 140. It can also contain an optional activation light filter 124 (which are sometimes called a pass filter, bandwidth filter, or similar terms). However, if light source 122 emits only a specific wavelength or narrow bandwidth that does not interfere with measurements of a fluorescing wavelength, activation light filter 124 is typically omitted.

As shown in FIG. 1, sample holder 130 is placed directly in the path of the excitatory light. The sample 132 held by sample holder 130 can be pre-mixed with a fluorophore. Alternatively, the interior walls of a sample holder can be pre-coated or attached with a fluorophore. Pre-coating of fluorophores onto sample holder surfaces can be achieved using any of several known techniques for attaching organic compounds to solid substrate surface. Fluorophores can be attached to porous matrices, gel-type materials, or other materials; or, they can be attached to the inner surfaces of the sample holder itself. Fluorophores can be attached to a solid substrate or the inner surfaces of the sample holder by means of spacer chains (e.g., linkers) that can optimize fluorophore orientation and accessibility.

As shown in FIG. 1, a light filter 142 with a relatively narrow (or “monochromatic”) bandwidth can be placed between the sample holder 104 and the photodetector 140 to ensure that emitted light that reaches photodetector 140 is limited to a relatively narrow fluorescing bandwidth. The use of various light filters that can be inserted into and removed from an accommodating slot can allow a variety of different interchangeable light filters to be used in the apparatus 100. This approach allows the use of different fluorophores that fluoresce at different wavelengths. For illustrative purposes, since several known zinpyr compounds such as ZP1 and ZP4 fluoresce at green wavelengths, FIG. 1 illustrates that light filter 142 and photodetector 140 operate at green wavelengths. Alternately, some types of photodetectors provide fluorescent emission intensity data over an entire spectrum, or over some significant portion of the spectrum. In such devices, correlations of zinc concentrations with fluorescent emission intensity can be based on either: (1) fluorescent intensity at a single specific wavelength, such as 530 nm green emissions when 480 nm blue excitation is used; or, (2) fluorescent intensity over a designated range of wavelengths, such as 520 to 540 nm; the major requirement in such cases is that the response curves for that fluorophore must have been determined, based on the same limits and parameters.

To reduce the incidence of misleading results that may be increased by unwanted effects, photodetector 140 should be positioned so that it will detect emitted light that leaves sample holder 130 at an angle substantially different from the beam of excitatory light. FIG. 1 indicates a right angle displacement between the two arrows that indicate the blue excitatory light beam and the green fluorescing light beam.

Photodetector 140 is typically wired, soldered, or otherwise electrically connected to an integrated circuit and/or microprocessor assembly 150. Generally, integrated circuit or microprocessor 150 is contained within the apparatus 100. The IC or microprocessor 150 typically generates a data signal that can be either (i) transferred to a computer, and/or (ii) converted into a display on a numerical panel on the apparatus. However, as will be recognized by those skilled in the art, other circuit and processing systems can be used if desired. For example, photodetector 140 can be selected to merely generate a voltage, resistance, capacitance, or other electronic value, and other components positioned outside the box can interpret that value and convert it into a data signal, numerical concentration, etc.

Integrated circuit or microprocessor 150 can be loaded with any desired combination of either or both of the following: (1) “fixed” instructions that cannot be readily altered by operators; and, (2) programmable capability that will allow the integrated circuit and/or microprocessor 150 to interact with a computer, in ways that an operator can control at will, by using software loaded into the computer. Such “fixed” instructions can include any combination of (i) algorithms, code, and other instructions that are permanently burned into integrated circuit or microprocessor 150 during manufacture, and (ii) additional “semi-permanent” code, such as code that is often referred to as “electronically programmable read-only memory” (i.e., EPROM). In general, EPROM and similar systems are often used to allow only authorized personnel to modify a software set, to reduce the risk that the software might be accidentally deleted, corrupted, etc.). Software that can be revised and updated, but only by qualified suppliers, is common among software programs that allow owners to obtain upgrades, drivers, patches, or other code modifications (collectively referred to herein as “updates”) over the Internet. Typically, these types of software updates are downloaded in the form of an executable file, which will have a command set that will cause the update package to insert the modified lines of code automatically into the prior software, after the update software has been downloaded by a user. Accordingly, if a zinc meter is coupled (using a USB or similar cable) to a computer that has Internet capability (even if only via a dial-up option), the computer can be provided with a relatively small “update” program that will allow the instruction set in the zinc meter to be periodically updated, as new and improved versions of the software become available over the course of successive years.

Alternately, many types of microprocessors have been developed with proprietary devices that have specialized hardware and access modes to ensure that the software can be revised only by authorized people or companies. These types of proprietary systems often use small plug-in devices, roughly comparable to fuses or circuit breakers but with memory arrays and multi-lead plug-in interfaces, which contain code that can be modified only by using specialized machines. These types of devices and systems are well known to people who specialize in designing, building, and programming “dedicated” microprocessor devices (often referred to by terms such as “programmable logic circuits”).

For zinc meters as disclosed herein, an already-known device called a spectrofluorimeter, available from Ocean Optics (www.oceanoptics.com), can be used to provide a number of the electronic and photodetector components of the machines described herein. Briefly, this type of device typically uses a diffraction grating or similar device that disperses light into spectrum, which is projected onto a linear “charge-coupled device” array. Ocean Optics also provides software that can be used to interact with those devices; however, more sophisticated software can be programmed and developed for particular uses.

Integrated circuit or microprocessor 150 can also be connected (e.g., via multi-lead cables or similar means) to the external USB port 110, and to any display devices 170 that may be mounted on the apparatus 100. In addition, a control button 180 (or any other suitable switch or activator device) can be wired into the electronic system in a manner that will trigger a measuring and data-sending operation each time the button is pressed.

If a pZn numerical system is used, it will display a number that will automatically accommodate a very wide range of possible values, where the first number in the pZn value indicates the exponent in a base 10 measuring system. As indicated above, this type of numerical system conveniently allows “p” values to accurately indicate any value within a huge range (for example, pH values that range from 1 to 14 can indicate acidity or alkalinity levels covering 14 orders of magnitude. Accordingly, a single display panel 170 would be required for such a system. As mentioned above, display panel 170 needs not be present in the apparatus 100. In such embodiments, data is typically displayed on an external display unit, e.g., a computer monitor.

If desired, integrated circuit or microprocessor 150 (or an accompanying computer) can be provided with memory capability to allow data from a series of measurements to be stored (in addition to any other desired information such as date and time of fluorescence measurement). This can provide backup and support capability, in case data is unexpectedly lost due to a power failure, computer failure, etc.

In some embodiments, an adjustment or calibration knob 190 is also provided on apparatus 100. The calibration knob 190 can be readily accessible on its surface or is protected beneath a covering device if desired. The calibration knob 190 allows periodic adjustment or calibration of the device using standard solutions having known concentrations of zinc ion. Periodic calibration allows the apparatus to remain reliable and accurate over a span of multiple years, despite factors that may gradually accumulate to a point where they would otherwise begin to affect accuracy (such as, for example, gradual dimming of the light-emitting bulb or diode, accumulation of dust or chemical films on optical surfaces, etc.).

In some aspects of the invention, the device or apparatus for measuring zinc ion concentration comprises:
a light source designed to emit an excitatory light at a first known wavelength that activates a fluorescent emission having a second and different wavelength by at least one selected fluorescent reporter probe that generates a different fluorescent response when bound to zinc ion;
at least one sample-holding component adapted to hold a liquid sample in a pathway of the excitatory light;
a photodetector for measuring fluorescent emission intensity generated by fluorescent reporter probe bound to zinc ion within the liquid sample; and
electronic means for creating a data signal that correlates with fluorescent emission intensity generated by fluorescent reporter probe bound to zinc ion in the liquid sample to the free zinc ion concentration.

In some embodiments, the apparatuses of the invention further comprise an enclosure and support component that supports the light source, sample-holding component, and photodetector. Such enclosure and support component can also provide a movable enclosure means that allows a liquid sample to be placed in and removed from the sample-holding component. Moreover the enclosure component is adapted to prevent ambient light from reaching the sample-holding component or the photodetector while the concentration of free zinc in the liquid sample is being measured.

In other embodiments, apparatuses of the invention display intensity generated by molecules (fluorophores) in the liquid sample as a pZn numerical value. As expected, in such embodiments, the apparatuses of the invention also include an electronic means for converting emission data to pZn value and conveying the pZn numerical value to the display component. Alternatively, apparatuses of the invention can include an means for conveying a pZn numerical value of the data signal to a computer.

Still in other embodiments, the excitatory light at a first known wavelength travels in a first pathway, and the fluorescent emission having a second and different wavelength travels to the photodetector in a second nonaligned pathway.

Yet in other embodiments, apparatuses of the invention further include means for detecting fluorescent emissions created by an assortment of different fluorogenic reagents, and a means for allowing an operator to select a suitable fluorogenic reagent for measuring the free zinc ion concentration.

Also disclosed herein is a kit for assembling such a device containing the components disclosed herein. Such components can be packaged in a way that instructs purchasers or users to assemble the kit into a working device.

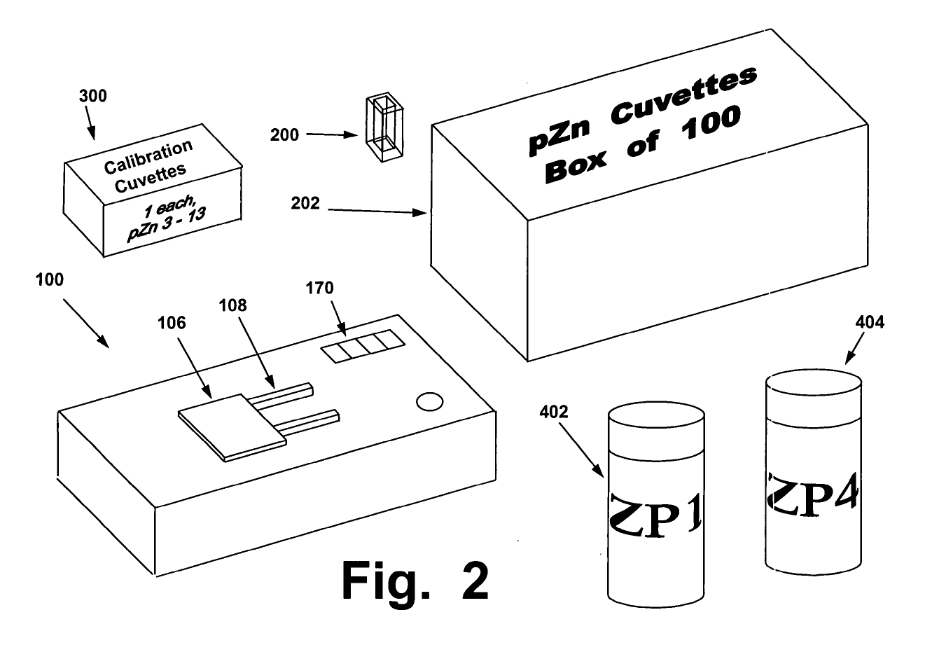

A complete system with additional accessories for measuring zinc ion levels is illustrated in FIG. 2. Such system can include metering device 100, a supply 202 of sample holders 200, and a supply of at least one fluorophore reporter probe, illustrated as bottles 402 and 404 of the “zinpyr” reagents ZP-1 and ZP-4 (unless sample holders are already coated with such reagents). In some embodiments, the complete system also comprises a set of “calibration sample holders” 300 that contain controlled mixtures with known concentrations of zinc ion. Such calibration sample holders can be sealed so that they will not change over time.

In addition, a complete system can also include one or more reagents that is capable of binding to one or more types of ions that might otherwise bind and/or react in undesired ways with one or more zinc-binding reagents. Transition metals that can be in a divalent (+2) oxidation state under normal conditions (e.g., iron, copper, lead, cadmium, mercury, nickel, etc.) can react with some of the zinc-binding fluorophore reagents mentioned above. Depending on the particular fluorophore that is being used, and the types of non-zinc ions that are present, such interference might lead to either: (1) “false positive” binding reactions, which might cause inaccurately high readings, as can occur when cadmium binds to reagents such as ZP1, or (2) “antagonistic” binding reactions (i.e., reactions that will occupy a zinc binding site without triggering a fluorescent change in the fluorophore), which might cause inaccurately low readings, as can occur when copper, mercury, or other ions bind to ZP1.

To prevent these types of unwanted reactions involving other divalent metals, a sample to be measured can be pretreated by using one or more chelating agents that will bind selectively and/or with greater affinity to divalent metal ions other than zinc to render the bound ions unable to bind to fluorophores. Chelating agents that bind to other divalent metals with much greater affinity and/or selectivity than zinc are well known to one skilled in the art, e.g., as certain types of ringed cyclam derivatives. In addition, certain types of specialized software programs (such as a program called MineQL) and databases (maintained and made available by organizations such as the National Institute for Standards and Technology (NIST) and the International Union of Pure and Applied Chemists (IUPAC)) are available. These software programs and databases can be used to screen and identify candidate compounds that are useful for sequestering non-zinc metal ions prior to measuring the zinc ion amount in a sample.

Methods are also disclosed herein for measuring free zinc ion concentrations in a sample of interest. Such methods typically include:
mixing a known quantity of a sample with a reagent mixture comprising a fluorophore receptor probe that will bind to zinc ion, if the zinc ion is present in the sample;
placing the mixture in a sample holder; and
determining the zinc ion concentration in the sample using the apparatus disclosed herein.

While the presence (or absence or a likelihood) of variety of clinical conditions can be determined using methods and apparatuses of the invention, the present inventor has found that such methods and apparatuses are particularly useful in determining the presence or the risk of prostate cancer in a subject. In such methods a fluid sample, such semen, expressed prostatic fluid, VB3 urine, or a mixture thereof, is obtained from the subject of interest and the amount of free zinc ion in the fluid sample is determined using the apparatus disclosed herein. Such analysis allows one skilled in the art to determine whether that particular subject has prostate cancer or has a higher risk in having prostate cancer.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method for determining the presence of prostate cancer or the risk for prostate cancer in an individual, said method comprising:
determining an amount of free zinc in a fluid sample of an individual using a zinc ion detection apparatus, wherein the fluid sample comprises semen, expressed prostatic fluid, VB3 urine, or a mixture thereof; and
determining the presence of prostate cancer or the risk for prostate cancer in the individual using the amount of free zinc determined from the fluid sample of the individual,
wherein the zinc ion detection apparatus determines the zinc ion concentration in the fluid sample excluding zinc ions bound to proteins, peptides, amino acids and the zinc ions in spermatozoa or endothelial cells that maybe present in the fluid sample, and wherein the zinc ion detection apparatus comprises:
a light source designed to emit an excitatory light at a first known wavelength that activates a fluorescent emission having a second and different wavelength by at least one selected fluorescent reporter probe that generates a different fluorescent response when bound to zinc ion;
at least one sample-holding component adapted to hold a liquid sample in a pathway of the excitatory light;
a photodetector for measuring fluorescent emission intensity generated by fluorescent reporter probe bound to zinc ion within the liquid sample; and
electronic means for creating a data signal that correlates with fluorescent emission intensity generated by fluorescent reporter probe bound to zinc ion in the liquid sample to the free zinc ion concentration.

2. The method of claim 1, wherein the zinc ion detection apparatus further comprises an enclosure and support component that supports the light source, sample-holding component, and photodetector, and that provides a movable enclosure means that allows a liquid sample to be placed in and removed from the sample-holding component, and that prevents ambient light from reaching the sample-holding component or the photodetector while the concentration of free zinc in the liquid sample is being measured.

3. The method of claim 1, wherein the electronic means correlates fluorescent emission intensity generated by molecules in the liquid sample as a pZn numerical value.

4. The method of claim 3, wherein the electronic means further comprises a display component and means for conveying the pZn numerical value to the display component.

5. The method of claim 3, wherein the electronic means further comprises means for conveying a pZn numerical value of the data signal to a computer.

6. The method of claim 1, wherein the excitatory light at a first known wavelength travels in a first pathway, and the fluorescent emission having a second and different wavelength travels to the photodetector in a second nonaligned pathway.

7. The method of claim 1, wherein the zinc ion detection apparatus further comprises means for detecting fluorescent emissions created by an assortment of different fluorogenic reagents, and a means for allowing an operator to select a suitable fluorogenic reagent for measuring the free zinc ion concentration.

8. The method of claim 1, wherein the zinc ion detection apparatus is a portable apparatus.

9. The method of claim 1, wherein said step of determining the amount of free zinc comprises:
placing the fluid sample from the individual into a container comprising a permeable barrier that isolates a fluorescent reporter probe from the fluid sample, wherein the permeable barrier allows the zinc that are not bound to proteins, peptides, amino acids and the zinc that are not in spermatozoa or endothelial cells that maybe present in the fluid sample to diffuse through and bind to the fluorescent reporter probe to produce a probe-bound zinc; and
measuring a fluorescence level of the probe-bound zinc.

10. The method of claim 1, wherein the fluid sample comprises prostatic fluid, seminal fluid, or a combination thereof.

11. The method of claim 1, wherein the fluid comprises expressed prostatic fluid.

12. The method of claim 1, wherein the fluorescent reporter probe comprises a ZnAF photoinduced electron transfer zinc (II) sensor.

13. The method of claim 1, wherein ZnAF PET zinc (II) sensor is ZnAF-2.
